(19) United States
(12) Patent Application Publication
(10) Pub. No.: US 2019/0290552 A1
(43) Pub. Date: Sep. 26, 2019

(54) ETHANOLIC COMPOSITIONS COMPRISING PERFUME

(71) Applicant: Conopco, Inc., d/b/a UNILEVER, Englewood Cliffs, NJ (US)

(72) Inventors: Martin Peter CROPPER, Birkenhead (GB); Craig Warren JONES, Prenton, Wirral, Merseyside (GB); Hailey KELSO, Chester, Cheshire (GB); James MERRINGTON, West Kirby, Wirral (GB)

(73) Assignee: Conopco, Inc., d/b/a UNILEVER, Englewood Cliffs, NJ (US)

(21) Appl. No.: 16/345,908

(22) PCT Filed: Oct. 17, 2017
(86) PCT No.: PCT/EP2017/076497

§ 371 Date: Apr. 29, 2019

(30) Foreign Application Priority Data
Nov. 3, 2016 (EP) 16197115.5

(51) Int. Cl. A61K 8/02; A61Q 13/00; A61K 8/34; A61K 8/58; A61K 8/25; A61Q 15/00

(52) U.S. Cl. CPC A61K 8/0279; A61Q 13/00; A61Q 15/00; A61K 8/585; A61K 8/25; A61K 8/34

(57) ABSTRACT

A composition for application to skin comprising: (i) at least 40 wt % ethanol, (ii) from 0.1 to 6 wt % perfume comprising non-polar perfume materials, (iii) optionally, at least 0.2 wt % surfactant, (iv) 0.1 to 6 wt % of porous microparticles comprising sol-gel derived material, the sol-gel derived material including a plurality of alkylsiloxy substituents and wherein the sol-gel derived material is obtained from: (a) at least one first alkoxysilane precursor having the formula: (R′O)₃—Si—(CH₂)—Ar—(CH₂)m-Si—(OR′)₃ (1) where n and m are individually an integer from 1 to 8, Ar is a single-, fused-, or poly- aromatic ring, and each R′ is independently a C₁ to C₅ alkyl group and (b) optionally, at least one second precursor having the formula: (2) where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the sol-gel derived material is swellable to at least 2.5 times its dry mass, when placed in excess acetone, whereby at least part of the perfume remains outside the microparticles. Also, a method of prolongation of perfume delivery from a perfumed composition comprising the steps of: (i) adding sol-gel derived silica microparticles as described above to an ethanolic composition comprising ethanol and perfume, comprising non-polar perfume components, dispersed or dissolved in the ethanol, and optionally surfactant; (ii) applying the composition to skin to deposit the microparticles onto the skin (iii) evaporating the ethanol to leave some perfume and the microparticles on the skin; absorbing at least part of the non-polar perfume components from the liquid into the microparticles, preferably such that the microparticles increase in weight by 20 to 80 wt %, and (iv) releasing perfume from the microparticles over a period of from up to 24 hours.

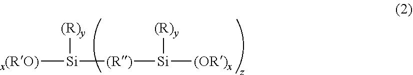

### TECHNICAL FIELD

This invention relates to ethanolic compositions, particularly those intended to be applied to skin, comprising perfume.

### BACKGROUND

Perfume containing microcapsules are used in many home and personal care products. Typically the microcapsule has a shell that fractures after the microcapsule has been deposited and dried out. Usually there is also free perfume in the composition to provide fragrance prior to rupture of the shell and possibly to provide a complimentary fragrance under some rupture conditions. Organic shell materials used in the prior art are well known to suffer from a lack of compatibility with ethanol which means that they are unsuitable for use in personal care compositions such as body sprays and deodorant sticks comprising significant levels of ethanol. There is a long standing need for an ethanol compatible microparticle based perfume release system.

Recently, a new type of organic inorganic hybrid sol gel microparticle has been disclosed in U.S. Pat. No. 8,367,793B2 and US 2010/0096334A1 (ABS Materials), and P. Edmiston; Organic-Inorganic Hybrids, Chem. Mater. 2008, 20, 1312-1321. Other silica sol gel materials have been disclosed for absorption of perfume but they do not swell as much and do not have the same selectivity for non-polar materials. See for example: WO 2015083836A1, and WO 2012088758A1.

In “Aroma Retention in Sol-Gel-Made Silica Particles” by Veith, Susanne R.; Pratsinis, Sotiris E.; Perren, Matthias; Journal of Agricultural and Food Chemistry (2004), 52(19), 5964-5971, the retention performance of aroma molecules from different chemical classes (e.g., alcohols, esters, aldehydes, and terpenes) by silica particles made by hydrolysis of tetra-Et orthosilicate is investigated. Since particle morphology, porosity, and pore size distribution can be controlled by the sol-gel preparation method, the influence of the nano confinement in the microporous matrix on aroma retention is studied as well as the effect of the initial aroma load of the particles. As the porosity is decreased, aroma molecules are entrapped more efficiently in the silica particles.

More recently in Cosmetics and Toiletries vol 128 No. 10 October 2013 “Swellable, Nanoporous Organosilica for extended and triggered release”; Paul L Edmiston investigated the use of nanoporous organosilica (Osorb from ABS materials) for extended release of volatile fragrances and the stimulated release of active ingredients. These materials swell rapidly with organic solvents. The animated organosilica consisted of polycondensed alkoxysilane precursors that contained a bridging organic group possessing an aryl ring. This aromatic group allows for pi-pi stacking, enabling the molecular self-assembly of the particles that cross link and thus comprise the matrix. The material may be ground to a powder. The organosilica was prepared using hexamethyldisilazane as post polymerisation derivatisation agent as described in C M Burkett, L A Underwood R S Volzer J A Baughman and P L Edmiston; organic inorganic hybrid materials that rapidly swell in non-polar liquids; nanoscale morphology and swelling mechanism. Chemistry of Materials 20(4) 1312-1321 (2008). The disclosure of perfume was limited to Rose extract diluted 1:20 in dichloromethane and added until the organosilica was fully swollen (5.5 mL/g). The dichloromethane was allowed to evaporate.

None of these prior art documents describes or suggests to use swellable organosilica for enhanced delivery of fragrance from ethanol based compositions, such as deodorants.

### SUMMARY OF THE INVENTION

According to a first aspect of the present invention there is provided a composition for application to skin comprising:
(i) at least 40 wt % ethanol,
(ii) from 0.1 to 6 wt % perfume comprising non-polar perfume materials,
(iii) optionally, at least 0.2 wt % surfactant,
(iv) 0.1 to 6 wt % of porous microparticles comprising sol-gel derived material, the sol-gel derived material including a plurality of alkylsiloxy substituents and wherein the sol-gel derived material is obtained from:
(a) at least one first alkoxysilane precursor having the formula:

(R′O)₃—Si—(CH₂)ₙ—Ar—(CH₂)ₘ—Si—(OR′)₃ (1)
where n and m are individually an integer from 1 to 8, Ar is a single-, fused-, or poly-aromatic ring, and each R′ is independently a C₁ to C₅ alkyl group and
(b) optionally, at least one second precursor having the formula:

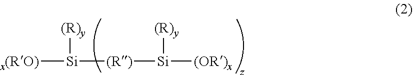

where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the sol-gel derived material is swellable to at least 2.5 times its dry mass, when placed in excess acetone,
whereby at least part of the perfume remains outside the microparticles.

In a spray the composition may be mixed with a propellant. In that case the levels of the components will be reduced due to the dilution effect of the propellant. Throughout this specification all composition amounts are references to the base composition, excluding any propellant that may also be present when it is in a container.

In one embodiment the plurality of alkylsiloxy groups have the formula:

—(O)_{w}—Si—(R₃)_{4-w} (3)

where each R₃ is independently an organic functional group and w is an integer from 1 to 3.

Preferably the non-polar perfume absorbed into the microparticles has a log Kow of greater than 2.8, preferably greater than 4.

The first alkoxysilane precursors of formula (1) are preferably selected from the group consisting of bis(trimethoxysilylethyl)benzene, 1,4-bis(trimethoxysilylmethyl)benzene and mixtures thereof.

Advantageously the microparticles have a volume average diameter in the composition of 10 to 100 microns, preferably 20 to 80 microns.

Preferably for a silkier feel on skin the microparticles have a microporous structure.

In a preferred embodiment the composition is a personal care composition comprising from 0.1 to 6 wt % perfume, excluding any propellant, whereby the majority of the perfume is not absorbed in the microparticles.

Also according to the invention there is provided a method of prolongation of perfume delivery from a perfumed composition comprising the steps of:
(i) adding sol-gel derived silica microparticles according to the first aspect to an ethanolic composition comprising ethanol and perfume comprising non-polar perfume components, dispersed or dissolved in the ethanol, and optionally surfactant;
(ii) applying the composition to skin to deposit the microparticles onto the skin,
(iii) evaporating the ethanol to leave some perfume and the microparticles on the skin;

absorbing at least part of the non-polar perfume components from the liquid into the microparticles, preferably such that the microparticles increase in weight by 20 to 80 wt %, and
(iv) releasing perfume from the microparticles over a period of from up to 24 hours.

The composition may be applied to skin in the form of a spray, through a spray nozzle. The composition may be an aerosol composition or a non-aerosol composition.

### DETAILED DESCRIPTION OF THE INVENTION

Throughout this specification references to percentages are to weight percentages unless the context demands otherwise.

A new type of organically linked silica sol gel microparticle having either a micro- or meso-porous structure has recently been developed as discussed in the background section herein. It differs from other silicas in that it is capable of being reversibly highly swollen by non-polar materials. These hybrid organic-inorganic materials comprise at least one type of organic bridging group that contains an aromatic segment that is flexibly linked to the alkoxysilane polymerisable ends. We have shown that by absorbing perfume from a perfumed composition it can control the release of the perfume from the composition, both in terms of intensity and duration; thus providing enhanced fragrance delivery.

It seems that when used in the inventive compositions and methods the sol-gel derived microparticles absorb a proportion of the total fragrance into the microparticle's 3-D network structure. Subsequently, because the absorption process is reversible the fragrance is able to diffuse slowly from the particles to provide a reservoir to extend fragrance longevity from a surface to which a composition comprising the fragranced particles has been delivered. This effect does not need any external mechanism to be applied such as solvent pulsing as used previously to flush an active material back out of the microparticle after it has been absorbed.

The sol-gel derived microparticle system also overcomes the known disadvantage of instability and premature release of perfume in the presence of ethanol that is associated with many know types of microcapsules. Deo compositions frequently comprise ethanol.

Typical synthetic methods for the sol-gels can be found in Chem. Mater. 2008, 20, 1312-1321; and U.S. Pat. No. 8,367,793 B2.

Suitable Silica sol gel derived microparticles are available from by ABS Materials Inc., Wooster, Ohio under the tradenames of Osorb™ or SilaFresh™. Osorb media has a microporous morphology in the dry state whereas SilaFresh™ media has a mesoporous structure. Neither product adsorbs water.

The sol-gel composition can be similar or identical to the swellable materials described in US2007/0112242 A1. For example, the sol-gel composition can include a plurality of flexibly tethered and interconnected organosilica particles having diameters on the nanometer scale. The plurality of interconnected organosilica particles can form a disorganized microporous array or matrix defined by a plurality of cross-linked aromatic siloxanes. The organosilica particles can have a multilayer configuration comprising a hydrophilic inner layer and a hydrophobic, aromatic-rich outer layer.

In the prior art it is said that the sol-gel composition has the ability to swell to at least twice its dried volume when placed in contact with a non-polar liquid. Without being bound by theory, it is believed that swelling may be derived from the morphology of interconnected organosilica particles that are crosslinked during the gel state to yield a nanoporous material or polymeric matrix. Upon drying the gel and following a derivatization step, tensile forces may be generated by capillary-induced collapse of the polymeric matrix. Stored energy can be released as the matrix relaxes to an expanded state when elements of the fabric treatment compositions disrupt the inter-particle interactions holding the dried material in the collapsed state. New surface area and void volume may then be created, which serves to further capture additional liquid that can diffuse into the expanded pore structure. Initial adsorption to the surface of the composition occurs in the non-swollen state. Further adsorption may then trigger matrix expansion which leads to absorption across the composition-water boundary. Pore filling may lead to further percolation into the composition, followed by continued composition expansion to increase available void volume. The mechanism for perfume prolongation in the present invention is not fully understood. It appears that before application to the skin the swelling behaviour of the prior art is not necessary for achievement of subsequent perfume prolongation in use.

The porous sol-gel composition is obtained from at least one first alkoxysilane precursor having the formula:

(RO)₃—Si—(CH₂)ₙ—Ar—(CH₂)ₘ—Si—(OR)₃ (1)

where n and m are individually an integer from 1 to 8, Ar is a single-, fused-, or poly-aromatic ring, such as a phenyl or naphthyl ring, and each R is independently a C₁ to C₅ alkyl, such as methyl or ethyl.

Exemplary first alkoxysilane precursors include, without limitation, bis(trialkoxysilylalkyl)benzenes, such as 1,4-bis(trimethoxysilylmethyl)benzene (BTB), bis(triethoxysilylethyl)benzene (BTEB), and mixtures thereof, with bis(triethoxysilylethyl)benzene being preferred.

In another aspect, the porous sol-gel composition is obtained from a mixture of the at least one first alkoxysilane precursor and at least one second alkoxysilane precursor, where the at least one second alkoxysilane precursor has the formula:

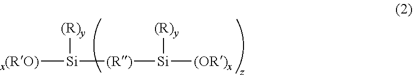

where x is 1, 2, 3 or 4; y is 0, 1, 2, 3; z is 0, 1; where the total of x+y+z is 4; R is independently an organic functional group; R′ is independently an alkyl group; and R″ is an organic bridging group, for example an alkyl or aromatic bridging group.

In one aspect, x is 2 or 3, y is 1 or 2 and z is 0 and R′ is a methyl, an ethyl, or a propyl group. In another aspect, R comprises an unsubstituted or substituted straight-chain hydrocarbon group, branched-chain hydrocarbon group, cyclic hydrocarbon group, or aromatic hydrocarbon group.

In some embodiments, each R is independently an aliphatic or non-aliphatic hydrocarbon containing up to about 30 carbons, with or without one or more hetero atoms (e.g., sulfur, oxygen, nitrogen, phosphorous, and halogen atoms) or hetero atom-containing moieties. Representative R's include straight-chain hydrocarbons, branched-chain hydrocarbons, cyclic hydrocarbons, and aromatic hydrocarbons and are unsubstituted or substituted. In some aspects, R includes alkyl hydrocarbons, such as C₁-C₃ alkyls, and aromatic hydrocarbons, such as phenyl, and aromatic hydrocarbons substituted with heteroatom containing moieties, such —OH, —SH, —NH₂, and aromatic amines, such as pyridine.

Representative substituents for R include primary amines, such as aminopropyl, secondary amines, such as bis(triethoxysilylpropyl)amine, tertiary amines, thiols, such as mercaptopropyl, isocyanates, such as isocyanopropyl, carbamates, such as propylbenzylcarbamate, alcohols, alkenes, pyridine, halogens, halogenated hydrocarbons or combinations thereof.

Exemplary second alkoxysilane alkoxysilane precursors include, without limitation, tetramethoxysilane, methyltrimethoxysilane, methyltriethoxysilane, dimethyldimethoxysilane, dimethyldiethoxysilane, phenyltrimethoxysiliane, aminopropyl-trimethoxysilane, (4-ethylbenzyl)trimethoxysilane, 1,6-bis(trimethoxysilyl)hexane, 1,4-bis(triethoxysilyl)benzene, bis(triethoxysilylpropyl)amine, 3-cyanopropyltrimethoxysilane, 3-sulfoxypropyltrimethoxysilane, isocyanopropyltrimethoxysilane, 2-(3,4-epoxycyclohexyl)ethyltrimethoxysilane, and Examples of suitable second precursors include, without limitation, dimethyldimethoxysilane, (4-ethylbenzyl)trimethoxysilane, 1,6-bis(trimethoxysilyl)hexane, 1,4-bis(trimethoxysilyl)benzene, tetramethoxysilane, methyltrimethoxysilane, methyltriethoxysilane, phenyltrimethoxysilane, with dimethyldimethoxysilane, (4-ethylbenzyl)trimethoxysilane, and phenyltrimethoxysilane being preferred.

Other examples of useful second precursors include, without limitation, para-trifluoromethylterafluorophenyltrimethoxysilane, (tridecafluoro-1,1,2,2-tetrahydro-octyl)trimethoxysilane; second precursors having a ligand containing —OH, —SH, —NH₂ or aromatic nitrogen groups, such as 2-(trimethoxysilylethyl)pyridine, 3-aminopropyltrimethoxysilane, 3-mercaptopropyltrimethoxysilane, and second precursors with protected amine groups, such as trimethoxypropylbenzylcarbamate.

In one aspect, the second alkoxysilane alkoxysilane precursor is dimethyldimethoxysilane, dimethyldiethoxysilane, phenyltrimethoxysilane or aminopropyltriethoxysilane.

The properties of the sol-gel derived composition can be modified by the second precursor. The second alkoxysilane precursor can be selected to produce sol-gel compositions having improved properties. In one aspect, the sol-gel derived compositions are substantially mesoporous. In one aspect, the sol-gel derived compositions contain less than about 20% micropores and, in one aspect, the sol-gel derived compositions contain less than about 10% micropores. In one aspect, the mesopores have a pore volume greater than 0.50 mL/g as measured by the BET/BJH method and in one aspect, the mesopores have a pore volume greater than 0.75 mL/gas measured by the BET/BJH method. In another aspect, the sol-gel derived composition generates a force upon swelling that is greater than about 200 N/g as measured by swelling with acetone in a confined system; in one aspect, the sol-gel derived composition generates a force upon swelling that is greater than about 400 N/g as measured by swelling with acetone in a confined system and in one aspect one aspect, the sol-gel derived composition generates a force upon swelling that is greater than about 700 N/g as measured by swelling with acetone in a confined system.

The sol-gel derived compositions may absorb at least 2.5 times the volume of acetone per mass of dry sol-gel derived composition. Examples of second precursors useful to effect the swellability of the sol-gel derived composition include dimethyldimethoxysilane, (4-ethylbenzyl)trimethoxysilane, 1,6-bis(trimethoxysilyl)hexane, 1,4-bis(trimethoxysilyl)benzene methyltrimethoxysilane, phenyltrimethoxysilane, with dimethyldimethoxysilane, (4-ethylbenzyl)trimethoxysilane, and phenyltrimethoxysilane being preferred.

The porous sol-gel compositions are obtained from an alkoxysilane precursor reaction medium, under acid or base sol-gel conditions, preferably base sol-gel conditions. In one aspect of the present invention, the alkoxysilane precursor reaction medium contains from about 100:00 vol:vol to about 10:90 vol:vol of the at least one first alkoxysilane precursor to the at least one second alkoxysilane precursor, in one aspect, and from about 20:80 vol:vol to about 50:50 vol:vol first alkoxysilane precursor to second alkoxysilane precursor. In one aspect, the alkoxysilane precursor reaction medium contains 100% of the at least one first alkoxysilane alkoxysilane precursor. The relative amounts of the at least one first alkoxysilane and the at least one second alkoxysilane alkoxysilane precursors in the reaction medium will depend on the particular alkoxysilane precursors and the particular application for the resulting sol-gel composition.

The reaction medium includes a solvent for the alkoxysilane precursors. In some aspects, the solvent has a Dimoth-Reichart solvatochromism parameter (ET) between 170 to 205 kJ/mol. Suitable solvents include, without limitation, tetrahydrofuran (THF), acetone, dichloromethane/THF mixtures containing at least 15% by vol. THF, and THF/acetonitrile mixtures containing at least 50% by vol. THF. Of these exemplary solvents, THF is preferred. The alkoxysilane precursors are preferably present in the reaction medium at between about 0.25M and about 1M, more preferably between about 0.4M and about 0.8M, most preferably about 0.5 M.

A catalytic solution comprising a catalyst and water is rapidly added to the reaction medium to catalyze the hydrolysis and condensation of the alkoxysilane precursors, so that a sol gel coating is formed on the particles. Conditions for sol-gel reactions are well-known in the art and include the use of acid or base catalysts. Preferred conditions are those that use a base catalyst. Exemplary base catalysts include, without limitation, tetrabutyl ammonium fluoride (TBAF), fluoride salts, including but not limited to potassium fluoride, 1,5-diazabicyclo[4.3.0]non-5-ene (DBN), and alkylamines, including but not limited to propyl amines, of which TBAF is preferred.

As noted above, acid catalysts can be used to form sol-gel coatings, although acid catalysts are less preferred. Exemplary acid catalysts include, without limitation, any strong acid such as hydrochloric acid, phosphoric acid, sulfuric acid and the like.

In one aspect, water is present in the reaction medium at an amount so there is at least one half mole of water per mole of alkoxysilane groups in the alkoxysilane precursors. In one aspect, temperatures at polymerization can range from between the freezing point of the reaction medium up to the boiling point of the reaction medium. And in one aspect, the temperature range is from about 4° C. to about 50° C.

After gellation, the sol-gel coating is preferably aged for an amount of time suitable to induce syneresis, which is the shrinkage of the gel that accompanies solvent evaporation. The aging drives off much, but not necessarily all, of the solvent. While aging times vary depending upon the catalyst and solvent used to form the gel, aging is typically carried out for about 15 minutes up to about 10 days. In one aspect, aging is carried out for at least about 1 hour and, in one aspect, aging is carried out for about 2 to about 10 days. In one aspect, aging temperatures can range from between the freezing point of the solvent or solvent mixture up to the boiling point of the solvent or solvent mixture. And in one aspect, the aging temperature is from about 4° C. to about 50° C. And in some aspects, aging is carried out either in open atmosphere, under reduced pressure, in a container or oven.

After gellation and aging have been completed, the sol-gel composition is rinsed using an acidic solution, with solutions comprising stronger acids being more effective. In one aspect, the rinsing agent comprises concentrations between 0.009 to 0.2% w/v acid in an organic solvent. Representative organic solvents include solvents for the alkoxysilane precursors, including solvents having a Dimoth-Reichart solvatochromism parameter (ET) between 170 to 205 kJ/mol. Suitable solvents for use with the base catalysts include, without limitation, tetrahydrofuran (THF), acetone, dichloromethane/THF mixtures containing at least 15% by vol. THF, and THF/acetonitrile mixtures containing at least 50% by vol. THF. Preferred rinse reagents, include without limitation, 0.01% wt:vol HCI or 0.01% wt:vol H2SO4 in acetone. In one aspect, the sol-gel composition is rinsed with the acidic solution for at least 5 min. And in one aspect, the sol-gel composition is rinsed for a period of time from about 0.5 hr to about 12 hr.

An alternative rinsing method is to use a pseudo-solvent system, such as supercritical carbon dioxide.

After rinsing, the sol-gel derived material is characterized by the presence of residual silanols. In one aspect, the silanol groups are derivatized with a reagent in an amount sufficient to stoichiometrially react with the residual silanols and prevent cross-linking that might otherwise occur between the residual silanol groups. Suitable derivatization reagents include, without limitation, reagents that have both one or more silanol-reactive groups and one or more non-reactive alkyl groups. The derivatization process results in the end-capping of the silanol-terminated polymers present within the sol-gel derived material with alkylsiloxy groups having the formula:

—(O)_{w}—Si—(R₃)₄ (3)

where each R₃ is independently an organic functional group as described above and w is an integer from 1 to 3.

One suitable class of derivatization reagents includes halosilanes, such as monohalosilane, dihalosilane and trihalosilane derivatization reagents that contain at least one halogen group and at least one alkyl group R₃, as described above. The halogen group can be any halogen, preferably Cl, FI, I, or Br. Representative halosilanederivatization reagents include, without limitation, chlorosilanes, dichlorosilanes, fluorosilanes, difluorosilanes, bromosilanes, dibromosilanes, iodosilanes, and di-iodosilanes. Exemplary halosilanes suitable for use as derivatization reagents include, without limitation, cynanopropyldimethyl-chlorosilane, phenyldimethylchlorosilane, chloromethyldimethylchlorosilane, (trideca-fluoro-1,1,2,2-tertahydro-octyl)dimethylchlorosilane, n-octyldimethylchlorosilane, and n-octadecyldimethylchlorosilane. And in one aspect, the halosilane derivatization reagent is trimethyl chlorosilane.

Another suitable class of derivatization reagents includes silazanes or disilazanes. Any silazane with at least one reactive group and at least one alkyl group R₃, as described above can be used. A preferred disilazane is hexamethyldisilazane.

The sol-gel derived composition is preferably rinsed in any of the rinsing agents described above to remove excess derivatization reagent, and then dried. Drying can be carried out under any suitable conditions, but preferably in an oven, e.g., for about 2 hours at about 60° C. to produce the porous, swellable, sol-gel derived composition.

In some aspects, the compositions contain a plurality of flexibly tethered and interconnected organosiloxane particles having diameters on the nanometer scale. The organosiloxane particles form a porous matrix defined by a plurality of aromatically cross-linked organosiloxanes that create a porous structure.

In some aspects, the resulting sol-gel compositions are hydrophobic, resistant to absorbing water, and absorb at least 2.5, even at least five and sometimes at least ten times the volume of acetone per mass of dry sol-gel derived composition. Without being bound by theory, it is believed that swelling is derived from the morphology of interconnected organosilica particles that are cross-linked during the gel state to yield a porous material or polymeric matrix. Upon drying the gel, tensile forces are generated by capillary-induced collapse of the polymeric matrix. This stored energy can be released as the matrix relaxes to an expanded state when a sorbate disrupts the inter-particle interactions holding the dried material in the collapsed state.

In one aspect, the resulting sol-gel composition contains a plurality of flexibly tethered and interconnected organosiloxane particles having diameters on the nanometer scale. The organosiloxane particles form a porous matrix defined by a plurality of aromatically cross-linked organosiloxanes that create a porous structure. In some aspects, the resulting sol-gel composition has a pore volume of from about 0.9 mL/g to about 1.1 mL/g and, in some aspects, a pore volume of from about 0.2 mL/g to about 0.6 mL/g. In some aspects, the resulting sol-gel composition has a surface area of from about 50 m²/g to about 600 m²/g and, in some aspects, a surface area of from about 600 m²/g to about 1000 m²/g.

In one aspect, the resulting sol-gel composition is hydrophobic, resistant to absorbing water, and swellable to at least 2.5 times its dry mass, when placed in excess acetone. In one aspect, the resulting sol-gel composition is hydrophobic, resistant to absorbing water, and swellable to at least five times its dry mass, when placed in excess acetone and, in one aspect, the sol-gel composition is swellable to at least ten times its dry mass, when placed in excess acetone.

Ethanolic Compositions

Preferred compositions are body sprays comprising mainly ethanol and perfume and deodorant sticks comprising a significant proportion of ethanol. Other ingredients may also be included in such compositions as is normal in the art. For example a body spray may further comprise a propellant if a pump dispenser is not used. Although not normally present an antiperspirant may also be included, again at normal levels.

The compositions of the present invention comprise greater than 25%, preferably greater than 50%, and more preferably greater than 65%, of C1 to C4 monohydric alcohol carrier fluid comprising ethanol, by weight of the total composition (excluding any volatile propellant present). The exclusion of volatile propellant during the calculation of the above values is equivalent to saying that the levels quoted relate the ‘base’ composition when the composition concerned comprises a volatile propellant. Within the base composition of aerosol compositions, it is further preferred that the alcohol carrier fluid is present at a level in the base composition of greater than 90% by weight, more preferably greater than 95% by weight.

The compositions of the invention preferably have a weight ratio of C1-C4 monohydric alcohol carrier fluid comprising ethanol to water of greater than 65:35, more preferably greater than 90:10. In certain particularly preferred compositions, notably aerosol compositions, the weight ratio of C1-C4 monohydric alcohol carrier fluid comprising ethanol to water is between 95:5 and 99:1. In other particularly preferred compositions, notably aerosol compositions, the weight ratio of C1-C4 monohydric alcohol carrier fluid comprising ethanol to water is greater than 99:1.

The monohydric alcohol carrier fluid comprising ethanol is preferably ethanol or isopropanol, with ethanol as the sole carrier fluid being most preferred.

Water is a preferred solubility promoter in compositions comprising a chelator that is in the form of a salt or acid salt having an inorganic cation or an organic cation formed from a water-soluble amine. The water serves as a solubility promoter by increasing the polarity of the total solvent system.

In compositions for use in roll-on, squeeze spray, or pump spray dispensers, the water is preferably present at a level of from 4 to 50% and more preferably at a level of from 15 to 40% by weight.

In aerosol compositions, the water is preferably present at less than 25%, preferably less than 10%, by weight of the base composition and is preferably used in combination with an organic amine solubility promoter. In aerosol compositions, it is preferred that the weight ratio of C1-C4 monohydric alcohol carrier fluid to water is greater than 65:35, more preferably greater than 90:10. Certain preferred aerosol compositions comprising water have a weight ratio of C1-C4 monohydric alcohol carrier fluid to water of 95:1 to 99:1 and an organic amine solubility promoter. Other preferred aerosol compositions have a weight ratio of C1-C4 monohydric alcohol carrier fluid to water of greater than 99:1 and particular organic amine and/or other solubility promoter(s) present (vide infra).

Compositions with relatively low levels of water can be of particular value in products applied to the human body. When such compositions contain relatively high levels of water, they can sometimes cause an undesirable wet sensation on application. Relatively low water level compositions can also be of benefit with regard to container choice: such compositions enable metal containers to be used with less risk of corrosion. A further benefit of compositions having relatively low water levels is their compatibility with additional hydrophobic components, for example fragrance components (see “Perfumery: practice and principles”, R. R. Calkin and S. Jellinek, [Wiley, 1994, p 171]).

Other materials that may be included in the compositions are organic amines, chelators, polyhydric alcohols and, for aerosol compositions, propellants. Antimicrobial agents may be used. Preferred anti-microbials are bactericides, in particular organic bactericides, for example quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred anti-microbials for use in the compositions of the invention are polyhexamethylene biguanide salts (also known as polyaminopropyl biguanide salts), an example being Cosmocil CQ™ available from Zeneca PLC, preferably used at up to 1% and more preferably at 0.03% to 0.3% by weight; 2′,4,4′-trichloro,2-hydroxy-diphenyl ether (triclosan), preferably used at up to 1% by weight of the composition and more preferably at 0.05-0.3%; and 3,7,11-trimethyldodeca-2,6,10-trienol (famesol), preferably used at up to 1% by weight of the composition and more preferably at up to 0.5%.

Phenolic Anti-Oxidants may also be included in the compositions. Preferred materials for incorporation into compositions of the invention are butylated hydroxytoluene (BHT) and butylated hydroxyanisole (BHA). Such agents are preferably used at 0.05% to 5%, more preferably 0.075% to 2.5%, and most preferably 0.1% to 1% by weight of the composition, excluding any volatile propellant present.

Certain sensory modifiers are further desirable components in the compositions of the invention. Emollients, humectants, volatile oils and non-volatile oils are all suitable classes of sensory modifiers. Examples of such materials include cyclomethicone, dimethicone, dimethiconol, isopropyl myristate, isopropyl palmitate, C12-C15 alcohol benzoate, PPG-3 myristyl ether, octyl dodecanol, C7-C14 isoparaffins, di-isopropyl adipate, isosorbide laurate, PPG-14 butyl ether, glycerol, hydrogenated polyisobutene, polydecene, phenyl trimethicone, dioctyl adipate, and hexamethyl disiloxane.

The perfume or free fragrance is an essential component in the compositions of the invention. Suitable materials include conventional perfumes, such as perfume oils and also include so-called deo-perfumes, as described in EP 545,556 and other publications. Levels of incorporation are up to 6% by weight, particularly from 0.1% to 3% by weight, excluding any volatile propellant present. A fragrance solubiliser is also a desirable component in many compositions. Such materials are emulsifiers that aid the dissolution/dispersion of a fragrance material in a composition. Preferred levels for incorporation are from 0.05% to 2%, preferably from 0.1% to 0.5%, by weight of the composition, excluding any volatile propellant present. These materials are of particular value when the ratio of water to C1 to C4 monohydric alcohol carrier fluid is greater than 25:75 and especially when it is greater than 35:65. Preferred materials are nonionic surfactants of HLB from 5 to 20 and particularly preferred materials include ethoxylated fatty alcohols, ethoxylated fatty acids, and ethoxylated oils, an example of the latter being PEG-40 hydrogenated castor oil.

Further additional components that may also be included are colourants, preservatives, for example C₁-C₃ alkyl parabens, and anticlogging agents, at conventional concentrations.

The composition may contain an antiperspirant active. Antiperspirant actives are preferably incorporated in an amount of from 0.5 to 50 wt %, particularly from 5 to 30 wt % and especially from 10% to 26 wt % of the composition. It is often considered that the main benefit from incorporating of up to 5 wt % of an antiperspirant active in a stick composition is manifest in reducing body odour, and that as the proportion of antiperspirant active increases, so the efficacy of that composition at controlling perspiration increases.

Antiperspirant actives for use herein are often selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Aluminium halohydrates are usually defined by the general formula:

Al₂(OH)ₓQy-wH₂O in which Q represents chlorine, bromine or iodine, x is variable from 2 to 5 and x+y=6 while wH₂O represents a variable amount of hydration. Especially effective aluminium halohydrate salts, known as activated aluminium chlorohydrates, are described in EP-A-6739 (Unilever N V et al).

Zirconium actives can usually be represented by the empirical general formula: ZrO(OH)₂ₙ₋nzB_{z}.wH₂O in which z is a variable in the range of from 0.9 to 2.0 so that the value 2n-nz is zero or positive, n is the valency of B, and B is selected from the group consisting of chloride, other halide, sulphamate, sulphate and mixtures thereof. Possible hydration to a variable extent is represented by wH₂O. Preferable is that B represents chloride and the variable z lies in the range from 1.5 to 1.87. In practice, such zirconium salts are usually not employed by themselves, but as a component of a combined aluminium and zirconium-based antiperspirant.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine. It is highly desirable to employ complexes of a combination of aluminium halohydrates and zirconium chlorohydrates together with amino acids such as glycine, which are disclosed in U.S. Pat. No. 3,792,068 (Luedders et al).

The invention will now be further described with reference to the following non-limiting examples.

Sol-Gel Materials

Two types of the sol-gel elastic materials were assessed: Osorb® and SilaFresh™ media (Table 1). Osorb media is distinguished by a microporous morphology in the dry state. SilaFresh media possess a mesoporous' structure. Neither product adsorbs water. The materials had been prepared using the methods described in Chem. Mater. 2008, 20, 1312-1321; and U.S. Pat. No. 8,367,793 B2. Both publications describe the synthesis. It is the processing conditions that determine whether the structure is micro- or meso-porous.

TABLE 1
Properties of Osorb ® and SilaFresh ™ Media

| Property | Osorb ® Media* | SilaFresh ™ Media* |
|---|---|---|
| Surface area, m²/g | 550 | 90 |
| Pore volume (dry), mL/g | 0.55 | 0.65 |
| Sebum capacity, mL/g | 3 | 4.5 |
| media | | |
| Pore size type | microporous | mesoporous |

*INCI name: Dimethicone/Phenyl Silsesquioxane/Phenyl Bis-Silsesquioxane Crosspolymer

Mesoporous material was observed to give a more pleasant silky feel when in contact with human skin. For that reason it may be preferred for compositions applied to skin.

### EXAMPLES

### Example 1

### Fragrance Release Kinetics of Full Fragrance from Ethanol

SilaFresh media was used to demonstrate the longevity of fragrances when applied from ethanol containing compositions.

In the examples larger cut means media sieved to be in the range 25 to 78 microns and smaller cut means media sieved to be in the range 17 to 56 microns.

Method: Two commercial fragrance containing deodorant compositions consisting of 2.0% v/v Fragrance A or B in ethanol were prepared. The fragrance components measured in Fragrances A and B are given in Table 2. SilaFresh media was loaded into each perfume containing composition at the following levels: 0.2% w/w, 0.8% w/w, and 2.0% w/w. Control compositions with no SilaFresh media were also evaluated. At these low levels of inclusion and in the presence of ethanol not all of the perfume from the composition was ever absorbed by the SilaFresh.

Direct injection GC-MS analysis was performed and the individual fragrance mixtures showed variable ratios of low boiling point compounds compared to the higher boiling point musky notes. Table 2 details the fragrance notes measured during the study.

TABLE 2
Fragrances measured

| | Fragrance Components Measured via. SPME-GCMS |
|---|---|
| Fragrance | (LogK_{ow}) |
| A | Limonene (3.4), Linalool (2.7), Linalyl Butyrate (4.1), |
| | Lilial (3.6), Methyl dihydrojasmonate (2.7), |
| | Oxacyclotetradecan-2-one (4.7), Ethylene brassylate (4.2). |
| B | Limonene (3.4), 3,7-Dimethyl-1,6-nonadien-3-ol (3.7), |
| | Thujopsene (4.8), Lilial (3.6), Methyl dihydrojasmonate (2.7), |
| | Ethylene brassylate (4.2). |

LogKow values are taken from the Good Scents website.

To begin a time course experiment, 0.3 mL of the composition comprising fragrance and silica microparticles was sprayed onto an 11 cm diameter Pyrex glass dish pre-equilibrated to 37° C.

Following the subsequent evaporation of the ethanol, the dishes were placed in a mechanically ventilated oven at 37° C. to approximate body temperature.

Headspace SPME-GC/MS using a PDMS/DVB fibre was used to measure the amount of fragrance in the gas phase at 1, 3, 5, 8 hr. Initially, the dishes were sealed in polypropylene container with a hole to allow a SPME fibre to be inserted. Disposable foil lids were used for headspace sampling. SPME sampling was done for 5 min at the temperature of the experiment. A cotton ball containing 50 μL hexadecane (b.p. 287° C.) was added as an internal standard.

Prior to each analysis 6 or 7 representative fragrance compounds as listed in Table 2 for each perfume were identified by direct injection of the liquid sample. These compounds were chosen to span a wide range of boiling points to follow the decay of the different notes of the fragrance.

GC/MS: A capillary HP-5 column was used. Selective ion monitoring was employed for detection. Peak areas relative to the internal standard were used to quantify the relative concentration over time.

Headspace Results: The SPME-GS/MS method was used to measure the fragrance profiles. The log of total fragrance concentration was then recorded over the time period of 0 to 8 hr and is given in Table 3.

TABLE 3
Log of Concentration of Total Fragrance v Time at 37° C.

| | | Time | Log Total |
|---|---|---|---|
| Fragrance | % SilaFresh | (hours) | Fragrance |
| A | 0 | 0 | 2.8 |
| | | 5 | −1.5 |
| | | 8 | −1.7 |
| | 0.2 | 0 | 2.6 |
| | | 5 | −1.5 |
| | | 8 | −1.3 |
| | 0.8 | 0 | 2.8 |
| | | 5 | −0.8 |
| | | 8 | −0.5 |
| | 2.0 | 0 | 2.0 |
| | | 5 | −0.1 |
| | | 8 | −0.7 |
| B | 0 | 0 | 2.0 |
| | | 5 | −1.5 |
| | | 8 | −2.0 |
| | 0.2 | 0 | 2.0 |
| | | 5 | −1.0 |
| | | 8 | −0.7 |
| | 0.8 | 0 | 2.5 |
| | | 3 | 0.0 |
| | | 5 | −1.0 |
| | 2.0 | 0 | 2.0 |
| | | 3 | 0.0 |
| | | 5 | 0.0 |
| | | 8 | 0.1 |

The results show that there was an extension of fragrance longevity, as measured by its concentration in the head space over time, particularly at the 0.8% and 2.0% w/w addition.

We believe the mechanism of perfume retention is important here. That is when the Osorb/ethanol/perfume are sprayed the ethanol starts to flash off quicker than loss of perfume and hence, the perfume becomes more and more concentrated with the Osorb i.e. essentially the particle becomes loaded with fragrance at this point and released slowly due to a diffusional process through the pore structure.

### Example 2

Addition of 2.0% w/w SilaFresh microparticles led to about 15 to 30 times more total measured fragrance in the headspace compared to the control at 5 hr for the Fragrances B and A respectively. This is recorded in Table 4.

TABLE 4
Headspace GC/MS Data for 5 Hours Post-Application

| | | Normalized Total Peak Area at 5 hr, | | | | |
|---|---|---|---|---|---|---|
| | Incubation | Percent SilaFresh ™ Addition | | | | Increase |
| Fragrance | Temp ° C. | 0% | 0.2% | 0.8% | 2.0% | w/2.0% |
| A | 40 | 16261 | 14977 | 115265 | 492255 | 30.0× |
| B | 37 | 4568 | 8336 | 8935 | 68676 | 15.3× |

Gravimetric Analysis: A Perkin Elmer Pyris 1 TGA was used to measure the mass of fragrance remaining over time. After taring the balance, 20 μL of ethanolic body spray formulation was added onto the sample pan. The mass was recorded over a 24 hr time period. Afterwards, the pan was placed in an oven at 120° C. for 4 hr to desorb any remaining fragrances and the final mass determined to confirm the dry mass of the SilaFresh microparticles in the applied volume. Separate gravimetric experiments were performed and corresponded with the headspace measurements. Using gravimetry, the total mass of fragrance is tracked over time to determine the rate of desorption over the period of the experiment. A reduction in the rate of mass decline is observed with the addition of SilaFresh microparticles to the formulation (Table 5).

TABLE 5
Slopes from the gravimetric data

| | Rate of Release | Rate of Release |
|---|---|---|
| | (mg/min) | (mg/min) |
| | Fast Evaporating | Slow Evaporating |
| SilaFresh | Lower Boiling Point | Higher Boiling Point |
| Media Added | Notes^{a} | Notes^{b} |
| 0% | 4.77 × 10⁻⁴ | 9.77. × 10⁻⁴ |
| 0.2% | 4.26 × 10⁻⁴ | 6.13. × 10⁻⁴ |
| 0.8% | 2.93. × 10⁻⁴ | 5.41. × 10⁻⁴ |
| 2.0% | 3.42. × 10⁻⁴ | 5.48. × 10⁻⁴ |

^{a}Slope fitted to the data for the rate over a time period of 0-300 minutes for the fast evaporating notes;
^{b}Slope fitted to the data for the rate over a time period of 500-1100 minutes.

There seems to be an optimum modification of rate of release at a ratio of perfume to perfume plus media of 0.8:2.8 or 28% loading.

### Example 7

### Malodour Reduction

Preparation of control formulation B Fragrance oil C (3.33g) was added to 96% ethanol (96.67g) and stirred thoroughly before use.

Preparation of Example 7: 1% Osorb (larger cut 25-78 μm) formulation—Fragrance oil C (3.33 g) and Osorb (1 g) were added to 96% ethanol (95.67 g) and stirred for 4-6 hours. Before use the formulation was stirred thoroughly because the Osorb settles.

Preparation of Olfaction Test Samples:

All samples were prepared in 125 mL amber jars for use in the olfaction test. Before addition of filter papers, each jar was rinsed with ethanol and dried. During a typical sniff test 10 uL of control or 1% Osorb formulation was added to a 30 mm filter paper within a jar and put into a vented fume hood for 5 minutes to eliminate ethanol. Samples were transferred to an oven which was set to 37° C. and samples were aged at both 3.5 hours and 24 hours. After the time periods, samples were removed from the oven and a model malodour (20 μL) was added to each filter paper within the jar and capped for 1 to 1.5 hours before each panellist begun the olfaction test.

Olfaction Test Protocol:

Two samples were sniffed, both with a model underarm malodour—one without Osorb and one with 1% Osorb (larger cut) and the sample with the highest malodour was recorded. Both 3.5 hour and 24 hour samples were sniffed and each pairing was duplicated. Each assessor received the sample sets in the same order, however each set was randomised. All tests were performed in olfaction panel room. Tests were run and the data was recorded on Compusense. All participants were pre-screened and 13 people completed the test.

A paired comparison test was set up using the data acquisition system Compusense. Compusense is a web based software, in which tests can be programmed, providing samples with a unique 3 digit code for each sample, thus ensuring that samples are presented blindly to the assessor in a randomised order. This ensures that variability and bias during presentation/evaluation was minimised. Test set up was based on the BSI standard 5495. Once testing was complete, the raw data was exported in to a csv file for analysis outside of Compusense.

Data analysis was conducted by collating the number of responses for each sample. Statistical validity was determined by the number of responses for a sample according to the published data tables (from the institute of perception). The level of significance was set at 0.05.

Olfaction Test—Malodour Intensity (n=26)

Samples aged at 37° C., model underarm malodour added just before capping for 1 h (larger cut Osorb—25-78 μm). 2×13 panellists (duplicate).

3.5 hour olfaction test showed no significant difference with the presence of Osorb.

24 hour olfaction test showed significant difference for the presence of Osorb and panellists perceived samples as less malodourous—this suggests Osorb is decreasing the intensity of the malodour and this was recognised at 24 hours by 20 panellists. The results are given in Table 11.

Table 11

TABLE 11

Number of panellists

| | | |
|---|---|---|
| Aging time (hours) | 3.5 | 24 |
| Example B (Control) | 15 | 20 |
| Example 7 (1% Osorb) | 11 | 6 |

### Example 8

### Malodour Control without Perfume Present

The test protocol was similar to Example 7, except that no perfume (fragrance) was used.

Ethanol Formulations:

Preparation of Control formulation C—96% ethanol/4% water was the control formulation i.e. no perfume or Osorb present.

Preparation of 1% Osorb (larger cut 25 to 78 μm) formulation D—Osorb (1 g) was added to 96%/4% water ethanol (99 g) and stirred for 4 to 6 hours. Before use the formulation was stirred thoroughly because Osorb settles.

Preparation of Olfaction Test Samples:

All samples were prepared in 125 mL amber jars for use in the olfaction test. Before addition of filter papers, each jar was rinsed with ethanol and dried. During a typical sniff test 10 μL of control or 1% Osorb formulation was added to a 30 mm filter paper within a jar and put into a vented fume hood for 5 minutes to eliminate ethanol. Samples were transferred to an oven which was set to 37° C. and samples were aged at both 3.5 hours and 24 hours. After the time periods, samples were removed from the oven and the model underarm malodour (20 μL) was added to each filter paper within the jar and capped for 1 to 1.5 hours before each panellist begun the olfaction test.

Olfaction Test Protocol:

Two samples were sniffed, both with body malodour—one without Osorb and one with 1% Osorb (larger cut) and the sample with the highest malodour was recorded. Both 3.5 hour and 24 hour samples were sniffed and each pairing was duplicated. Each assessor received the sample sets in the same order, however each set was randomised. All tests were performed in olfaction panel room. Tests were run and the data was recorded on Compusense. All participants were pre-screened and 15 people completed the test providing an n=30 data points.

The results are given in Table 12.

TABLE 12

Number of panellists

| Aging time (hours) | 3.5 | 24 |
|---|---|---|
| Example C (Control) | 16 | 16 |
| Example D (1% Osorb) | 14 | 14 |

The 3.5 hours olfaction test for malodour reduction when no fragrance is present showed no significant difference with Osorb present. The 24 hour olfaction test for malodour reduction without fragrance also showed no significant difference to the control. From this it is concluded that in order to derive a benefit for reducing malodour with Osorb a fragrance must be present as in Example 7; which suggests that the Osorb is not simply adsorbing the malodour but that the fragrance may be altering the structure of the Osorb to allow the malodour to be adsorbed and hence reduced perceptually.

### Example 9

### Effect of Ratio of Fragrance to Osorb on Free v Adsorbed Fragrance

Table 13 shows the level of fragrance not absorbed into Osorb in ethanol. The baseline is Fragrance D comprising non-polar components in ethanol; we also analysed Fragrance D levels in filtered samples made using the large and small cut Osorb materials with different fragrance amounts.

There is no reduction of Fragrance D in ethanol when Osorb is introduced, suggesting the solvent out-competes the Osorb in this case. This supports the hypothesis that fragrance only interacts strongly with Osorb when the ethanol is evaporated.

TABLE 13

| | | Particle | | | | |
|---|---|---|---|---|---|---|
| | | size | | 95% | | |
| | Osorb, | range, | Fragrance, | ethanol | | |
| Sample | % | μm | % | in water | Average | 95% |
| 9.A | 0 | N/A | 3.3 | To 100% | 5.56E+09 | 2.92E+08 |
| 9.1 | 0.5 | 25-78 | 3.3 | To 100% | 5.52E+09 | 7.00E+07 |
| 9.2 | 1 | 25-78 | 3.3 | To 100% | 6.28E+09 | 9.81E+07 |
| 9.3 | 5 | 25-78 | 3.3 | To 100% | 6.05E+09 | 8.47E+08 |
| 9.4 | 0.5 | 17-56 | 3.3 | To 100% | 5.82E+09 | 3.91E+08 |
| 9.5 | 1 | 17-56 | 3.3 | To 100% | 6.08E+09 | 2.47E+08 |
| 9.6 | 5 | 17-56 | 3.3 | To 100% | 5.90E+09 | 5.19E+08 |

Fragrance D level was assessed by direct injection GCMS. The result shown is the sum of the areas of all peaks found in this measurement.

### Example 10

### Measurement of Particle Size Distribution (PSD) of the Silica Microparticles with Ethanol or Ethanol/Fragrance Mixture

Three samples (10.1, 10.2 and 10.3) were prepared that contained 1% of the same particle size Osorb, the 96% ethanol/4% water mixed used previously and 3.3% of Fragrance D comprising non-polar components. For comparison, we also prepared a sample of 1% Osorb in 96% ethanol/4% water mix without any perfume/fragrance (10.A). Composition details are given in Table 14.

TABLE 14

| Material | Level, % |
|---|---|
| Fragrance D | 3.3 |
| Osorb | 1.0 |
| Water | 3.8 |
| Ethanol | 91.9 |

After storage at 20° C. and agitation to eliminate any aggregation all four samples then had their average diameters measured in microns using light scattering and the results are summarised in Table 15.

TABLE 15

| Sample Name | d (0.1) | d (0.5) | d (0.9) |
|---|---|---|---|
| 10.1:1% Osorb + Fragrance D | 13.129 | 61.460 | 111.798 |
| 10.2:1% Osorb + Fragrance D | 33.745 | 71.828 | 163.135 |
| 10.3:1% Osorb + Fragrance D | 34.803 | 68.133 | 132.713 |
| 10.A:1% Osorb | 34.064 | 65.513 | 106.306 |

Table 15 shows no major differences between the first three, fragrance-containing, results and the last fragrance-free (control) one. This suggests that there is no significant change in PSD on exposure to fragrance.

1. A composition comprising:
(i) at least 40 wt % ethanol,
(ii) from 0.1 to 6 wt % perfume comprising non-polar perfume materials,
(iii) optionally, at least 0.2 wt % surfactant,
(iv) 0.1 to 6 wt % of porous microparticles comprising sol-gel derived material, the sol-gel derived material including a plurality of alkylsiloxy substituents and wherein the sol-gel derived material is obtained from:
(a) at least one first alkoxysilane precursor having the formula:
(R′O)₃—Si—(CH₂)ₙ—Ar—(CH₂)ₘ—Si—(OR′)₃ (1)
where n and m are individually an integer from 1 to 8, Ar is a single-, fused-, or poly-aromatic ring, and each R′ is independently a C₁ to C₅ alkyl group and
(b) optionally, at least one second precursor having the formula: 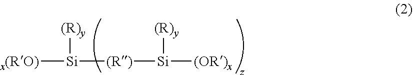
where x is 1, 2, 3 or 4; y is 0, 1, 2, 3; z is 0, 1; the total of x+y+z is 4; each R is independently an organic functional group; each an R′ is independently a C₁ to C₅ alkyl group and R″ is an organic bridging group, where the sol-gel derived material is swellable to at least 2.5 times its dry mass, when placed in excess acetone, whereby at least part of the perfume remains outside the microparticles.

2. The composition according to claim 1 wherein the plurality of alkylsiloxy groups have the formula:
—(O)_{w}—Si—(R₃)_{4-w} (3)
where each R₃ is independently an organic functional group and w is an integer from 1 to 3.

3. The composition according to claim 1 wherein the non-polar perfume absorbed into the microparticles has a log K_{0w} of greater than 2.8, preferably greater than 4.

4. The composition according to claim 1 wherein the first alkoxysilane precursors of formula (1) are selected from the group consisting of bis(trimethoxysilylethyl)benzene, 1,4-bis(trimethoxysilylmethyl)benzene and mixtures thereof.

5. The composition according to claim 1 wherein the microparticles have a volume average diameter in the composition of 10 to 100 microns, preferably 20 to 80 microns.

6. The composition according to claim 1 wherein the microparticles have a microporous structure.

7. The composition according to claim 1 further comprising from 0.1 to 6 wt % perfume whereby the perfume is not absorbed in the microparticles.

8. A method of prolongation of perfume delivery from a perfumed composition comprising the steps of:
(i) adding sol-gel derived silica microparticles according to claim 1 to an ethanolic composition comprising ethanol and perfume comprising non-polar perfume components, dispersed or dissolved in the ethanol, and optionally surfactant;
(ii) applying the composition to skin to deposit the microparticles onto the skin,
(iii) evaporating the ethanol to leave some perfume and the microparticles on the skin; absorbing at least part of the non-polar perfume components from the liquid into the microparticles, preferably such that the microparticles increase in weight by 20 to 80 wt %, and
(iv) releasing perfume from the microparticles over a period of from up to 24 hours.

9. The method according to claim 8 wherein the composition is applied to skin in a spray form, through a spray nozzle.

10. The method according to claim 9, wherein the composition is an aerosol composition.

11. The method according to claim 9, wherein the composition is a non-aerosol composition.

12. The composition according to claim 1 wherein the composition can be applied to skin.

13. The composition according to claim 1 wherein the composition is a personal care composition.
